(19) United States
(12) Patent Application Publication
(10) Pub. No.: US 2008/0138395 A1
(43) Pub. Date: Jun. 12, 2008

(54) Pharmaceutical Composition for the Prevention and Control of Cancer

(76) Inventor: Treusch Gernot, Empuria Brava (ES)

Correspondence Address: Thomas & Raring, P.C., 536 GRANITE AVENUE, RICHMOND, VA, 23226, US

(21) Appl. No.: 11/815,909

(22) PCT Filed: Feb. 14, 2006
(86) PCT No.: PCT/EP2006/001338

§ 371 Date: Aug. 9, 2007

(30) Foreign Application Priority Data
Feb. 14, 2005 (DE) 202005002324.8

(51) Int. Cl. A61K 9/127; A61K 38/06; A61P 35/00; A61K 38/44

(52) U.S. Cl. 424/450; 514/18; 424/94.4

(57) ABSTRACT

A pharmaceutical composition for the prevention and control of cancer containing, as pharmaceutically active substances, reduced glutathione and/or S-methyl glutathione and/or S-ethyl glutathione and/or S-acetyl glutathione and/or S-phosphoric glutathione and/or monomethyl glutathione ester and/or monoethyl glutathione ester and/or monoacetyl glutathione ester and/or monophosphoric glutathione ester and/or isopropyl glutathione ester, and at least one compound of the group consisting of betalains and the aglycones thereof.

It is known to use reduced glutathione or certain thiol derivatives of glutathione in combination with anthocyans or anthocyanidins among others for cancer control (EP 0 545 972 B1) In this combination, the pharmaceutical effect of the glutathione compound is enhanced synergistically by anthocyan or anthocyanidin.

It has now been found that, surprisingly, the combination of reduced glutathione or certain thiol compounds of glutathione with betalains or the aglycones thereof promises to have a much more pronounced effect against cancer than the previously known glutathione/anthocyan combination. In contrast to the assumption suggested by the prior art, the effect is especially distinct if the additional use or presence of anthocyans or anthocyanidins in the combination is dispensed with altogether.

The invention, therefore, proposes a pharmaceutical composition for the control of cancer comprising reduced glutathione and/or S-methyl glutathione and/or S-ethyl glutathione and/or S-acetyl glutathione and/or S-phosphoric glutathione, as well as at least one compound of the group consisting of betalains and the aglycones thereof as pharmaceutically active substances; any further active substances possibly present not belonging to the group of anthocyans or the aglycones thereof.

The subject matter of the invention, moreover, is the use of reduced glutathione and/or S-methyl glutathione and/or S-ethyl glutathione and/or S-acetyl glutathione and/or S-phosphoric glutathione, in combination with at least one compound of the group consisting of betalains and the aglycones thereof, preferably without the joint use of anthocyans or the aglycones thereof, for therapy, including dietetic preventive treatment of the human or animal body against cancer.

Glutathione (gamma glutamyl cysteinyl glycin) is a tripeptide of the three amino acids: glutamic acid, cystein, and glycin and is present in most human and mammal cells in its reduced form (G-SH). It structural formula is indicated in EP 0 545 972 B1.

In the composition according to the invention, reduced glutathione may be replaced fully or partly by certain thiol derivatives of glutathione, namely by the mono methyl thio ester, the mono ethyl thio ester, the mono acetyl thio ester, and/or the mono phosphoric acid thio ester of glutathione. The structural formula of these derivatives are likewise set forth in EP 0 545 972 B1.

In some applications, thiol derivatives of glutathione have the advantage of possessing greater bioavailability compared with reduced glutathione because of their great capability of permeation through biological membranes and also because with them the SH group of glutathione, important for the therapeutic effect, is protected all the way through the biological compartments up to the location where its effect is desired.

Certain glutathione esters with which esterification occurs at the carboxyl group of the glycyl radical rather than at the thiol group may be used as an alternative or in addition to the thiol derivatives of glutathione. These are monomethyl glutathione ester, monoethyl glutathione ester, monoacetyl glutathione ester, monophosphoric glutathione ester, and/or isopropyl glutathione ester. These derivatives, too, are more lipophilic than G-SH and, therefore, better able to permeate cell membranes.

Betalains are flower and fruit pigments derived from batalamic acid. Among them are the red violet betacyans from beetroot (beta vulgaris) and the yellow betaxanthins from the cactus family. Typical representatives of betacyans are betanin, isobetanin, prebetanin, and isoprebetanin. The aglycone of betanin is betanidine. Typical representatives of betaxanthins are vulgarxanthin I and vulgarxanthin II. Betalains primarily are obtained by isolation from the corresponding flowers and fruit; various tests permit a clear distinction from anthocyans to be seen, cf. the manual by Schweppe: Handbuch der Naturfarbstoffe. Vorkommen, Verwendung, Nachweis Landsberg: ecomed 1972, pages 407 to 409.

According to the invention, a composition is preferred in which the active substances are reduced glutathion and/or S-acetyl glutathion as well as betanin and/or betanidine. A method of preparing S-acetyl glutathion is disclosed in U.S. Pat. No. 2,760,956. Betanin also called “Beetenrot” (C₂₄H₂₆N₂O₁₃) is admitted for use as a food and cosmetics pigment. It is obtained from red beet and beetroot, respectively.

The composition according to the invention may contain such additional active substances as FAD (flavine-adenine-dinucleotide) and/or genistein obtained, for example, from a soybean source.

Another additional active substance in the composition according to the invention especially is peroxide dismutase (SOD), preferably at a daily dose of from 500 mg to 1500 mg.

The composition according to the invention may be used for all kinds of cancer and metastases, the forms of supply being for oral, nasal baccal, or sublingual administration and either by injection, infusion or as suppositories Further galenic improvement may be achieved by including the pharmaceutically effective agents in liposomes and/or cyclodextrins.

The toxicity of the active substances can be reduced in per se known manner by means of such substrates and their effectiveness enhanced by controlled release at precisely the location of the tumor or metastasis.

The effect of reduced glutathion (G-SH) and anthocyans and betacyans on tumor cell lines, specifically H69: human, small cell lung carcinoma, was examined by in vitro studies, both individually and with various combinations of the ingredient. G-SH and betalain were used in the form of dry extract from beta vulgaris (product: Betatom by Messrs. Sprüh-Atom Schumann & Sohn GmbH, Karlsruhe) and, for purposes of comparison, anthocyans in the form of dry extract from cassis (black currant) and vaccinium (blueberry). The tumor cells were sown in vitro in DMEM with 10% FCS. The final concentration of G-SH, Betatom, cassis and vaccinium, the substances added, was 1 mM. Apoptosis was measured with an Annexin testing system. All tests were repeated once. The effect on H69 tumor cells may be gathered from the table below. After treatment of the cells for ten days, the number both of apoptotic and defective cells was clearly higher in comparison with untreated controls. The combination of G-SH and Betatom without the presence of anthocyans (cassis and vaccinium) had the significantly greatest effect Table 1 sets forth the test results.

TABLE 1
Apoptotic and defective H69 tumor cells after 10 days of treatment

| | | | | | | | Cassis + | Betatom + | Betatom + | | |
|---|---|---|---|---|---|---|---|---|---|---|---|
| | | | | | Vaccinium + | Cassis + | Vaccinium + | Cassis + | Vaccinium + | Betatom + | |
| | GSH | Cassis | Vaccinium | Betatom | GSH | GSH | GSH | GSH | GSH | GSH | Control |
| apoptotic cells | 21.9 | 26.5 | 28.7 | 48.9 | 34.6 | 35.4 | 63.0 | 65.6 | 66.7 | 86.6 | 27.3 |
| (% of total cells) | | | | | | | | | | | |
| variation compared | −5 | −0.7 | 1.4 | 21.7 | 8.2 | 7.4 | 35.8 | 38.4 | 39.5 | 59.3 | |
| with control (%) | | | | | | | | | | | |
| defective cells | 37.9 | 45.2 | 45.4 | 58.0 | 51.3 | 54.3 | 61.0 | 68.7 | 72.5 | 88.9 | |
| (% of total cells) | | | | | | | | | | | |
| variation compared | 8.9 | 16.2 | 16.3 | 28.9 | 22.2 | 25.2 | 32.0 | 39.6 | 43.5 | 59.8 | |
| with control (%) | | | | | | | | | | | |

A typical preparation according to the invention for cancer control is available in the form of tablets for oral administration, each of them containing 200 mg of glutathion and 200 mg of Betatom as the exclusive active substances. They are administered at dosages of from 5 to 10 tablets per day.

While the invention has been described with reference to specific embodiments thereof, it will be understood that numerous variations, modifications and additional embodiments are possible, and all such variations, modifications, and embodiments are to be regarded as being within the spirit and scope of the invention.

What is claimed is:

1. A pharmaceutical composition for the prevention and control of cancer comprising:
a pharmaceutically active substance selected from the group consisting of reduced glutathione, S-methyl glutathione, S-ethyl glutathione, S-acetyl glutathione, S-phosphoric glutathione, monomethyl glutathione ester, monoethyl glutathione ester, monoacetyl glutathione ester, monophosphoric glutathione ester, and isopropyl glutathione ester, and mixtures thereof; and
at least one compound selected from the group consisting of the betalaines and the aglycones thereof.

2. The pharmaceutical composition as claimed in claim 1, wherein any other active substances that the composition may include do not belong to the group of anthocyan and the aglycones thereof.

3. A pharmaceutical composition for the prevention and control of cancer, comprising:
a pharmaceutically active substance selected from the group consisting of reduced glutathione, S-methyl glutathione, S-ethyl glutathione, S-acetyl glutathione, and S-phosphoric glutathione, and mixtures thereof; and
a compound selected from the group consisting of the betalains and the aglycones thereof.

4. The composition as claimed in claim 2 wherein the pharmaceutically active substance is selected from the group consisting of reduced glutathione, S-methyl glutathione, S-ethyl glutathione, S-acetyl glutathione, and S-phosphoric glutathione, and mixtures thereof; and
further comprising a compound selected from the group consisting of betacyans and the aglycones thereof.

5. The composition as claimed in claim 3, further comprising a compound selected from the group consisting of betacyans and the aglycones thereof.

6. The composition as claimed in claim 4, wherein the pharmaceutically active substance is selected from the group consisting of reduced glutathione and S-acetyl glutathione, and mixtures thereof; and
a compound selected from the group consisting of betanin and betanidin and mixtures thereof.

7. The composition as claimed in claim 1 further comprising peroxide dismutase.

8. The composition as claimed in claim 2 further comprising peroxide dismutase.

9. The composition as claimed in claim 4 further comprising peroxide dismutase.

10. The composition as claimed in claim 6 further comprising peroxide dismutase.

11. The composition as claimed in claim 1 wherein the pharmaceutically active substance is included completely or partly in liposomes and/or cyclodextrins as molecular substrates.

12. Use of a pharmaceutical composition as claimed in claim 1 or the pharmaceutically active substances thereof in combination with one another for treating a human or animal body against cancer.

13. The use as claimed in claim 12 without including the use of anthocyans and the aglycones thereof.
